(19) United States
(12) Patent Application Publication
(10) Pub. No.: US 2025/0134434 A1
(43) Pub. Date: May 1, 2025

(54) WEARABLE SENSOR HAVING AN ADHESIVE, AND A MANUFACTURING METHOD

(71) Applicants: André Filipe Marques-Smith, New Castle, DE (US); Ana Montero Horas, New Castle, DE (US); Catia Figueiredo, New Castle, DE (US); Goncalo Narciso, New Castle, DE (US); Joana Neto, New Castle, DE (US)

(72) Inventors: André Filipe Marques-Smith, New Castle, DE (US); Ana Montero Horas, New Castle, DE (US); Catia Figueiredo, New Castle, DE (US); Goncalo Narciso, New Castle, DE (US); Joana Neto, New Castle, DE (US)

(21) Appl. No.: 18/925,636

(22) Filed: Oct. 24, 2024

(30) Foreign Application Priority Data
Oct. 30, 2023 (GB) 2316545.9

(51) Int. Cl. A61B 5/259; A61B 5/28; A61B 5/291; A61B 5/296; C09J 183/06

(52) U.S. Cl. CPC A61B 5/259; A61B 5/28; A61B 5/291; A61B 5/296; C09J 183/06; A61B 2562/125; C09J 2479/02

(57) ABSTRACT

A skin-conformable and compact wearable sensor for monitoring surface physiological and/or surface brain signals of the wearer. The wearable sensor comprises a polymer adhesive. The adhesive polymer composition may include a silicone polymer and an amine-based polymer. The adhesive polymer composition may include from 1 to 10 wt. % amine-based polymer by weight of the adhesive polymer composition.

### CROSS-REFERENCES TO RELATED APPLICATIONS

This application claims the benefit of Great Britain Patent Application Number 2316545.9 filed on Oct. 30, 2023, the entire disclosure of which is incorporated herein by way of reference.

### FIELD OF THE INVENTION

The present techniques generally relate to a wearable sensor and manufacturing method for the wearable sensor. In particular, the present techniques provide a skin-conformable and compact wearable electronic apparatus for monitoring surface physiological and/or surface brain signals of the wearer and a manufacturing method for the apparatus. For example, the wearable electronic apparatus may be used to monitor electroencephalogram (EEG), electromyography (EMG) and/or electrocardiogram (ECG or EKG) signals.

### BACKGROUND OF THE INVENTION

Good quality ambulatory brain recordings are difficult to obtain because current techniques for monitoring human brain activity non-invasively are extremely susceptible to motion artefacts. Although devices exist which enable the collection of brain data outside clinical or laboratory environments, these devices require their wearer to refrain from making any head or body movements in order to acquire interpretable data. As such, it is currently difficult-without resorting to surgery-to monitor human brain activity effectively during most human behaviors. Solving this problem would, for instance, enable patients with neurological conditions to be monitored remotely, without interfering with their daily lives.

The present applicant has therefore identified the need for an improved sensor for monitoring physiological and/or brain activity data of users.

### SUMMARY OF THE INVENTION

The present techniques provide an adhesive composition which may be used, for example, on part of a skin-contacting sensor. In such a case, the adhesive composition may be used to improve the connectivity between a skin-contacting component of a sensor and skin, which may thereby improve the quality of sensed signals.

Thus, in a first approach of the present techniques, there is provided an adhesive polymer composition comprising: a silicone polymer; and an amine-based polymer. The adhesive polymer composition comprises from 1 to 10 wt. % amine-based polymer by weight of the adhesive polymer composition.

The term “adhesive polymer composition” is used interchangeably herein with the term “adhesive composition”.

Advantageously, the adhesive polymer composition comprising a silicone polymer and an amine-based polymer means that the adhesive will adhere well to a user's skin. In particular, the adhesive polymer composition may be easily applied and removed from a user's skin. The adhesive polymer composition may be cleaned and reapplied several times without significantly losing its adhesive properties.

The silicone polymer and/or amine-based polymer may be capable of forming a substantially continuous film layer after curing.

The silicone polymer may comprise an elastomer and a curing agent. That is, the silicone polymer may be provided in two parts, and a curing process of the silicone polymer may only start when a curing agent is added to the silicone polymer base. It will be understood by a person skilled in the art that the silicone polymer, or polymer mixture, prior to the curing process will be uncured, and that after the curing process the silicone polymer may be crosslinked, suitably via a curing agent, to form a crosslinked polymer network in the final adhesive polymer composition. For the avoidance of doubt, by “uncured” is meant that the silicone polymer has not yet reacted with a curing agent (or otherwise) to form a crosslinked polymer network. The term “uncured adhesive composition” is herein used interchangeably with “adhesive polymer mixture” and “uncured adhesive polymer mixture”.

Thus, the present techniques extend to an adhesive polymer mixture for forming an adhesive polymer composition, the adhesive polymer mixture comprising: a silicone polymer; and an amine-based polymer, wherein the adhesive polymer mixture comprises from 1 to 10 wt. % amine-based polymer by weight of the adhesive polymer mixture.

Advantageously, providing the silicone polymer and curing agent in two parts allows other components to be mixed with the elastomer base before a curing process starts, ensuring even distribution of any other components. The silicone polymer may preferably be inert and non-toxic, to ensure the polymer composition is suitable for contact with human or animal skin. The silicone polymer may be any suitable material. The silicone polymer may be polydimethylsiloxane (PDMS). PDMS is an example of an elastomer, and it will be understood that any other suitable elastomers may be used, as long as they are also inert and non-toxic. Other non-limiting examples of silicone-containing polymers which may be particularly suitable for forming a film layer include polysiloxane polyamides and polysiloxane polyureas.

The amine-based polymer may reduce or inhibit cross-linking bonds between the silicon base polymer and the curing agent for the silicone base polymer, creating heterogeneous cross-linking in the silicon polymer. The adhesive adheres to skin by van der Waals interactions.

In some cases, the silicone polymer may be polydimethylsiloxane.

The amine-based polymer may be any suitable material. For example, the amine-based polymer may be any of: polyethylenimine (PEI), including polyethylenimine PEI-derivatives such as ethoxylated polyethylenimine (PEIE), aminoethylaminopropyltrimethoxysilane (AEPS), and/or polyamidoamine (PAMAM). These may be used alone or in a mixture of two or more.

In some cases, the amine-based polymer may be polyethylenimine and/or ethoxylated polyethylenimine (PEIE).

In some cases, the amine-based polymer may be polyethylenimine (PEI).

In some cases, the amine-based polymer may be ethoxylated polyethylenimine (PEIE).

As noted above, the adhesive polymer composition comprises from 1 wt. % to 10 wt. % amine-based polymer by weight of the adhesive polymer composition. The adhesive polymer composition may comprise the amine-based polymer in a range from 1 wt. % to 2 wt. %, preferably in a range from 1.2 wt. % to 1.5 wt. %, more preferably in a range from 1.2 wt. % to 1.45 wt. %, more preferably in a range from 1.2 to 1.4 wt. %, most preferably in a range from 1.22 wt. % to 1.4 wt. %, by weight of the adhesive composition.

The adhesive polymer composition may comprise the amine-based polymer in a range from 1.2 to 1.5 wt. % by weight of the adhesive composition.

The adhesive polymer composition may comprise the amine-based polymer in a range from 1.2 to 1.4 wt. % by weight of the adhesive composition.

The adhesive polymer composition may comprise from 1 wt. % to 10 wt. % polyethylenimine (PEI) by weight of the adhesive polymer composition. The adhesive polymer composition may comprise polyethylenimine (PEI) in a range from 1 wt. % to 2 wt. %, preferably in a range from 1.2 wt. % to 1.5 wt. %, more preferably in a range from 1.2 wt. % to 1.45 wt. %, more preferably in a range from 1.2 to 1.4 wt. %, most preferably in a range from 1.22 wt. % to 1.4 wt. %, by weight of the adhesive composition.

The adhesive polymer composition may comprise polyethylenimine (PEI) in a range from 1.2 to 1.5 wt. % by weight of the adhesive composition.

Reference to “polyethylenimine (PEI)” includes PEI-derivatives such as ethoxylated polyethylenimine (PEIE).

As such, the adhesive polymer composition may comprise from 1 wt. % to 10 wt. % ethoxylated polyethylenimine (PEIE) by weight of the adhesive polymer composition. The adhesive polymer composition may comprise ethoxylated polyethylenimine (PEIE) in a range from 1 wt. % to 2 wt. %, preferably in a range from 1.2 wt. % to 1.5 wt. %, more preferably in a range from 1.2 wt. % to 1.45 wt. %, more preferably in a range from 1.2 to 1.4 wt. %, most preferably in a range from 1.22 wt. % to 1.4 wt. %, by weight of the adhesive composition.

The adhesive polymer composition may comprise ethoxylated polyethylenimine (PEIE) in a range from 1.2 to 1.5 wt. % by weight of the adhesive composition.

The adhesive polymer composition may be derived from an uncured adhesive composition (or polymer mixture). Thus, in a second approach of the present techniques, there is provided an uncured adhesive composition (or polymer mixture) comprising: a silicone polymer; a curing agent; and an amine-based polymer, wherein the adhesive polymer composition comprises from 1 to 10 wt. % amine-based polymer by weight of the adhesive polymer composition.

The features described above with respect to the first approach apply equally to the second approach and therefore, for the sake of conciseness, are not repeated.

The curing agent may be any suitable curing agent, and may depend on the silicone polymer.

In a particular example, the silicone polymer may be uncured polydimethylsiloxane and/or the curing agent may be the curing agent for polydimethylsiloxane and/or the amine-based polymer is polyethylenimine (PEI), including polyethylenimine PEI-derivatives such as ethoxylated polyethylenimine (PEIE). By “the” curing agent for polydimethylsiloxane is meant any suitable curing agent (as per above).

The curing agent may be used in any suitable amount. The adhesive polymer composition may comprise a curing agent in a range from 0.5 wt. % to 20 wt. %, preferably in a range from 1 wt. % to 15 wt. %, more preferably in a range from 2 wt. % to 10 wt. %, more preferably in a range from 4 wt. % to 8 wt. %, most preferably in a range from 4 wt. % to 6 wt. %, by weight of the adhesive composition.

The adhesive polymer composition may comprise less than 10 wt. % of a curing agent by weight of the adhesive composition.

The adhesive polymer composition may comprise about 5 wt. % of a curing agent by weight of the adhesive composition.

Advantageously, lower levels of curing agent may be used compared to known compositions.

In a third approach of the present techniques, there is provided a method for manufacturing an adhesive polymer composition, the method comprising: obtaining an adhesive polymer mixture by mixing a silicone polymer and an amine-based polymer; and curing the adhesive polymer mixture to obtain a cured adhesive polymer composition. The adhesive polymer mixture, after cure, may be used to form a film layer on a wearable sensor or components of a wearable sensor, to enable the sensor to better adhere to skin.

The features described above with respect to the first approach apply equally to the third approach and therefore, for the sake of conciseness, are not repeated.

Curing the adhesive polymer mixture may comprise at least one thermal curation step.

The at least one thermal curation step may take place at a predetermined curation temperature over a predetermined curation period. Controlling a temperature of a thermal curation step ensures that the curing process is controlled and therefore, along with controlling a ratio of a curing agent in the adhesive polymer composition, allows control over the ratio of cured to uncured polymer chains in the cured adhesive polymer composition. In turn, control over the ratio of cured to uncured polymer chains means that the properties of the adhesive polymer composition can be controlled. For example, a larger number of cured chains results in a more solid and less adhesive polymer, whereas less cured chains result in a softer and more adhesive polymer composition. These are important properties, as the softness and adhesiveness of the adhesive polymer composition determine how well the adhesive polymer composition conforms to a user's skin and thus how well the sensor as a whole conforms to a user's skin and consequently functions.

The at least one thermal curation step may comprise at least two thermal curation steps. A sequence of thermal curation steps may help ensure that the cured adhesive polymer composition has the required properties.

The at least two thermal curation steps may comprise a first thermal curation step taking place at a first predetermined curation temperature over a first predetermined curation period, and a second thermal curation step taking place at a second predetermined curation temperature over a second predetermined curation period.

In a fourth approach to the present techniques, there is provided a wearable sensor for monitoring physiological and/or brain signals, the sensor comprising: a thin flexible substrate comprising a skin-contacting surface, wherein the skin-contacting surface is at least partially coated in the adhesive polymer composition described herein.

In some cases, the adhesive polymer composition forms a thin film layer on the skin-contacting surface.

The wearable sensor may comprise at least one electrode pad for monitoring physiological and/or brain signals. Preferably, the wearable sensor may comprise at least two electrode pads, where one electrode pad is used as a reference and another electrode pad is used to sense a signal.

In cases where the adhesive polymer composition forms a thin film layer, the adhesive film layer may comprise at least one opening that corresponds to a position of the at least one electrode pad, so that the electrode pad is still able to contact skin. Coating the flexible substrate in an adhesive film layer ensures an even and comfortable fit of the sensor for a user.

The wearable sensor may comprise circuitry coupled to the at least one electrode pad. The circuitry may all be contained in a flexible (or rigid) PCB (see the readout electronics module described below with reference to the Figures). The at least one electrode pad may interface with the circuitry/readout electronics module via a connector. In some cases, the at least one electrode pad may be directly coupled to the circuitry.

In some cases, the at least one electrode pad may be at least partially coated in a coating formed of a conductive polymer composition. The term “conductive polymer composition” is used interchangeably herein with the term “electrically-conductive polymer composition”.

Advantageously, the adhesive film layer may be of substantially similar thickness as the conductive polymer electrode coating that is applied to the at least one electrode pad. This ensures that the sensor fits evenly to a user's skin and ensures that the fit of the sensor is skin conformal. Advantageously, an even and skin conformal fit ensures that no motion artefacts are introduced to an electrophysiological signal measured by the electrode pad by movement of a user. Additionally, coating the flexible substrate in an adhesive can ensure that application of the sensor to skin is straightforward and user-friendly.

The conductive polymer composition is suitable for providing an interface between the at least one electrode pad and a user's skin. That is, the conductive polymer composition may reduce an electrode-skin impedance, such that an electrode/electrode pad that is coated with the conductive polymer composition is able to better measure electrophysiological signals from a user's skin. Thus, the conductive polymer composition may be used to provide dry electrodes. The dry electrodes may be fixedly and electrically coupled to the electrode pad(s) of the wearable sensor, such that during use, the dry electrodes contact with skin of a human or animal wearer/user of the wearable sensor. The term “dry electrode” is used herein to mean electrodes which can be applied or adhered to skin without the use of any liquids or gels. For example, commonly used electrodes for EEG signal sensing may require a conductive electrolyte gel to be provided between the electrode and the skin, but gel-based electrodes cannot be self-applied and may not be suitable on skin having hair or/and for prolonged use. The dry electrodes described herein do not require the use of such a gel, and are advantageously able to conform to skin.

The term “dry electrode” is used interchangeably herein with the terms “polymer electrode”, “electrode”, “conductive polymer electrode” and “electrically-conductive polymer electrode”.

The conductive polymer composition may comprise: an electrically-conductive material; at least one additive; and a silicone polymer.

The conductive polymer composition is suitable for providing an interface between an electrode pad (which may be part of a sensor or wearable apparatus) and a user's skin. That is, the conductive polymer composition may reduce an electrode-skin impedance, such that an electrode/electrode pad that is coated with the conductive polymer composition is able to better measure electrophysiological signals from a user's skin.

Advantageously, a dry electrode formed using the conductive polymer composition may be dry, thin (e.g. <1 mm) and flexible, which enables the dry electrode to conform to a user's skin without requiring a liquid or gel electrolyte interface. Thus, the conductive polymer composition may be used to form a conductive polymer dry electrode that leaves little or no residue on a user's skin, can be reapplied several times and is comfortable for a user to wear.

As noted above, the conductive polymer composition comprises an electrically-conductive material. The electrically-conductive material is used in the conductive polymer composition to impart conductivity to the composition. The electrically-conductive material may be used without particular limitation as long as it has electron conductivity without causing chemical change. The electrically-conductive material may be a conductive allotrope of carbon, such as, for example, graphene, carbon nanotubes (CNTs), and graphite such as natural graphite and artificial graphite. The carbon may be doped or undoped. The carbon may be particulate carbon. The electrically-conductive material may be any of: carbonaceous materials such as carbon black, acetylene black, ketjen black, channel black, furnace black, lamp black, thermal black and carbon fiber; metal powder, metal fibers or metal nanoparticles such as copper, nickel, aluminum and silver; conductive whiskers such as zinc oxide and potassium titanate; conductive metal oxides such as titanium oxide; electrically-conductive polymers; conductive powders; and polyphenylene derivatives. These may be used alone or in a mixture of two or more.

In cases where the electrically-conductive material of the conductive polymer composition is provided by a carbon material, the carbon material may be a high conductivity carbon material. An example high conductivity carbon material is carbon black or particulate carbon black. However, it will be understood that this is a non-limiting example of a high conductivity carbon material.

The conductive polymer composition may comprise particulate carbon as the electrically-conductive material, and the particulate carbon may be in a range from 5 wt. % to 20 wt. % by weight of the electrically-conductive polymer composition. The particulate carbon may be present in a range from 12 wt. % to 18 wt. %, preferably in a range from 13 wt. % to 17 wt. %, more preferably in a range from 14 wt. % to 17 wt. %, by weight of the conductive polymer composition.

The particulate carbon may be present in an amount of about 15 wt. % or 16 wt. % by weight of the conductive polymer composition.

The particulate carbon may be present in an amount of about 14.9 wt. % or 15 wt. % by weight of the conductive polymer composition. The particulate carbon may be present in an amount of about 16 wt. % or 16.1 wt. % by weight of the conductive polymer composition.

The particulate carbon may be in any suitable form. The particulate carbon may be in the form of nanoparticles. By “nanoparticles” is meant particles having an average diameter in the range of nanometers (nm). Particulate carbon nanoparticles may have an average diameter up to 900 nm, such as up to 800 nm, such as up to 700 nm, such as up to 600 nm, such as up to 500 nm, such as up to 400 nm, such as up to 300 nm, such as up to 200 nm, such as up to 100 nm. Particulate carbon nanoparticles may have an average diameter ranging from 5 to 500 nm, such as from 10 to 300 nm, such as from 20 to 250 nm, such as from 30 to 200 nm, such as from 40 to 150 nm, such as from 50 nm to 100 nm.

As noted above, the conductive polymer composition comprises at least one additive. The at least one additive may be, for example, a surfactant. The electrically-conductive polymer composition may comprise a surfactant to improve the miscibility of the composition and/or to improve mechanical properties of the composition. The surfactant may be any suitable surfactant. For example, the surfactant may be an ionic or non-ionic surfactant. Preferably, the surfactant may be a non-ionic surfactant. The surfactant may be selected from any one of: those sold under the tradename Triton (commercially available from Dow), such as Triton X-100; those sold under the tradename Tween (commercially available from Croda), such as Tween 80, Tween 20, Tween 40, Tween 60 and/or mixtures thereof. These may be used alone or in a mixture of two or more. The surfactant may comprise Triton X-100 (also known as 2-[4-(2,4,4-trimethylpentan-2-yl) phenoxy] ethanol). The surfactant may comprise Tween 80 (also known as polyoxyethylene (20) sorbitan monooleate). However, it will be understood that these are non-limiting examples of a surfactant and that other surfactants may be used which have substantially similar properties.

In some cases, the at least one additive may be Triton X-100, and the Triton X-100 may be present in a range from 1 wt. % to 30 wt. %, preferably in a range from 1 wt. % to 20 wt. %, more preferably in a range from 5 wt. % to 15 wt. %, for example about 10 wt. % such as 10 wt. %, by weight of the conductive polymer composition.

In some cases, the at least one additive may be Tween 80, and the Tween 80 may be in a range from 1 wt. % to 30 wt. %, preferably in a range from 1 wt. % to 20 wt. %, more preferably in a range from 5 wt. % to 15 wt. %, for example about 10 wt. % such as 10 wt. %, by weight of the conductive polymer composition.

As noted above, the conductive polymer composition comprises a silicone polymer. The silicone polymer may comprise an elastomer base and a curing agent. That is, the silicone polymer may be provided in two parts, and a curing process of the silicone polymer may only start when a curing agent is added to the silicone polymer base. It will be understood by a person skilled in the art that the polymer mixture (or composition) prior to the curing process will be uncured, and that after the curing process the silicone polymer may be crosslinked, suitably via a curing agent, agent to form a crosslinked polymer network in the final conductive polymer composition. For the avoidance of doubt, by “uncured” is meant that the silicone polymer has not yet reacted with a curing agent (or otherwise) to form a crosslinked polymer network. The term “uncured composition” is herein used interchangeably with “uncured composition”, “polymer mixture” and “uncured polymer mixture”.

Thus, the present techniques extend to a polymer mixture for forming an electrically-conductive polymer composition, the polymer mixture comprising an electrically-conductive material; at least one additive; a silicone polymer; and a curing agent.

Advantageously, providing the silicone polymer and curing agent in two parts allows other components to be mixed with the elastomer base before a curing process starts, ensuring even distribution of any other components. The silicone polymer may preferably be inert and non-toxic, to ensure the conductive polymer composition is suitable for contact with human or animal skin. The silicone polymer may be any suitable material. The silicone polymer may be polydimethylsiloxane (PDMS). PDMS is an example of an elastomer, and it will be understood that any other suitable elastomers may be used, as long as they are also inert and non-toxic. For example, other non-limiting silicone-based elastomers include natural rubbers, and silicone.

On the proviso that a silicone polymer is present, other polymer materials may also be used without particular limitation as long as they have electron conductivity without causing chemical change. For example, non-limiting examples include polyurethanes.

As noted above, the conductive polymer composition comprises at least one additive. Thus, in some cases, the conductive polymer composition may comprise, at least, a first additive and a second additive. The first additive may be a surfactant, as noted above. The second additive may be a material which provides the conductive polymer composition with improved electrical conductivity properties. For example, the second additive may be ethylene glycol.

In cases where the second additive is ethylene glycol, the ethylene glycol may be in a range from 1 wt. % to 30 wt. %, preferably in a range from 1 wt. % to 20 wt. %, more preferably in a range from 3 wt. % to 15 wt. %, most preferably in a range from 6 wt. % to 8 wt. %, for example about 7 wt. % such as 7 wt. %, by weight of the conductive polymer composition.

As noted above, the electrically-conductive polymer composition may be used to improve the connectivity and conductivity between a skin-contacting component of a wearable sensor, and skin of a human or animal. Preferably, an electrical skin impedance of the conductive polymer composition (at 30 Hz) may be in a range from 100 kΩ to 2000 kΩ, preferably in a range from 100 kΩ to 1000 kΩ, more preferably in a range from 50 kΩ to 500 kΩ, most preferably in a range from 10 kΩ to 200 kΩ.

In an fifth approach of the present techniques, there is provided a method for manufacturing a wearable sensor for monitoring physiological and/or brain signals, the wearable sensor having a flexible substrate, the method comprising: coating at least part of the flexible substrate in an adhesive polymer mixture, the mixture comprising a silicone polymer and an amine-based polymer, wherein the adhesive polymer mixture comprises from 1 to 10 wt. % amine-based polymer by weight of the adhesive polymer mixture; and curing the adhesive polymer mixture to obtain a cured adhesive polymer coating.

The features described above with respect to the first to fourth approaches apply equally to the fifth approach and therefore, for the sake of conciseness, are not repeated.

The method may further comprise patterning a surface of the conductive polymer mixture, prior to curing, to obtain an increased surface area of the cured conductive polymer (i.e. of the cured coating).

### BRIEF DESCRIPTION OF THE DRAWINGS

Implementations of the present techniques will now be described, by way of example only, with reference to the accompanying drawings, in which:

FIG. 1A is a plan view of an example layout of a sensor for measuring surface physiological and/or surface brain signals;

FIG. 1B shows the sensor of FIG. 1A which is coated with an adhesive;

FIG. 2A shows a top and side perspective view of an example pattern that may be applied to the dry electrodes of FIG. 1B;

FIG. 2B shows a side view of an example pattern that may be applied to the dry electrodes of FIG. 1B;

FIG. 3 is a flowchart showing the steps involved in producing a conductive polymer composition for use as a dry electrode;

FIG. 4 is a flowchart showing the steps involved in producing the adhesive polymer composition;

FIG. 5 is a diagram showing an overview of the process for coating the F-PCB of the sensor with an adhesive; and

FIG. 6 is a block diagram of a system comprising the sensor and a readout electronics device.

### DETAILED DESCRIPTION OF THE PREFERRED EMBODIMENTS

Broadly speaking, embodiments of the present techniques provide a skin-conformable electrode array and compact wearable electronic apparatus for monitoring surface physiological and/or surface brain signals of the wearer. In particular, the present techniques provide flexible, dry electrodes which may be used, for example, to provide skin-contacting electrodes of a wearable sensor. The dry electrodes may be fixedly and electrically coupled to electrodes of a wearable sensor, such that during use, the dry electrodes contact with skin of a human or animal wearer/user of the wearable sensor.

Two causes of motion artefacts in electroencephalography are displacement of sensors relative to the skin and/or interruption or alteration of the quality of electrical contact between the skin and the sensor, and cable sway introducing electrical noise into analogue signals.

The present techniques solve the problem by providing an adhesive composition which may be used, for example, on part of a skin-contacting sensor. In such a case, the adhesive composition may be used to improve the connectivity between a skin-contacting component of a sensor and skin, which may thereby improve the quality of sensed signals.

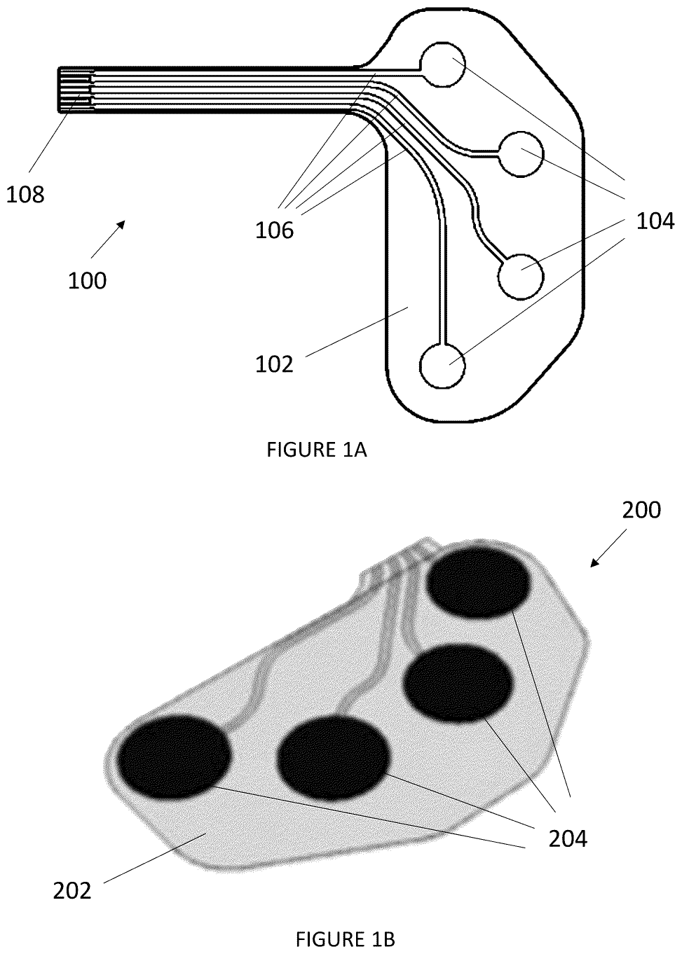

FIG. 1A is a plan view of an example layout of a sensor for measuring surface physiological and/or surface brain signals. The sensor 100 may comprise a substrate, such as, for example, a flexible printed circuit board (F-PCB) 102 on which at least one electrode pad 104 and at least one conductive track 106 are deposited. Preferably, the wearable sensor may comprise at least two electrode pads, where one electrode pad is used as a reference and another electrode pad is used to sense a signal. The sensor may be used for electrophysiological measurements such as EEG, ECG and/or EMG (which are the measurements most susceptible to motion-induced noise) or measurement of any other surface electrical signal from the body including, but not limited to, electro-oculogram (EOG), electro-gastrogram (EGG), electro-spinogram (ESG) or electro-olfactogram (EOFG). When the sensor is provided on a user's head, the at least one electrode pad may sense surface brain signals. In this case, the at least one electrode pad may be able to provide (directly or indirectly) information on the cognition, emotional state, or disease indicators. The at least one conductive track 106 may transmit measurements from the at least one electrode pad to a connector 108. In the example shown in FIG. 1A, the sensor 100 of the present techniques is advantageously not affected by cable sway. This is because circuits and connections between electrical elements of the apparatus are made through conductive tracks 106 deposited onto the flexible circuit board 102 itself, which moves with the skin. In contrast, existing devices use cables, which move relative to the skin and thus introduce motion artefacts into the signal. The sensor 100 may also comprise a connector 108 on the F-PCB 102. The connector 108 may be used to connect the sensor 100 to an apparatus and/or readout electronics for receiving a physiological signal that is measured by the sensor 100.

The substrate may be thin and flexible. The substrate may have a thickness in a range from 0.04 to 0.3 mm. For example, the substrate may have a thickness of 0.08 mm. The substrate may be made from flexible materials, and may comprise a polymer and/or polyimide and/or metal conductive tracks (e.g. copper) with a thickness in a range from 10 to 25 μm, for example, copper with a thickness of 18 μm. At least part of the substrate may have an immersion gold surface finish. When the substrate is a flexible printed circuit (F-PCB), the substrate may comprise the following layers: a polyimide substrate, copper, an electroless nickel immersion gold (ENIG) finish, and a polyimide coverlay (i.e. solder mask). A total thickness of a F-PCB stack may be between 0.3 mm to 0.5 mm.

The copper layer may have a finished thickness of 18 μm, the thin electroless nickel immersion gold (ENIG) finish may have a thickness of 3 rack units (U″) and the polyimide coverlay (i.e. solder mask) may have a thickness in a range from 5 to 20 μm, preferably in a range from 10 to 15 μm, for example, about 13 μm such as 12.5 μm or 13 μm. The overall F-PCB substrate stack may have a thickness in a range from 0.04 to 0.3 mm, for example, a thickness of 0.08 mm.

The at least one electrode pad 104 may be a gold electrode pad, which is highly conductive and highly resistant to corrosion. Additionally or alternatively, the at least one electrode pad 104 may be made from any other suitable highly conductive material, such as stainless steel, iridium, titanium, silver or another suitable metal, alloy or other material. The at least one electrode pad 104 may be placed at any suitable position on the F-PCB 102. When there is more than one electrode pad 104, the electrode pads 104 may be arranged in any suitable configuration. For example, the location of the electrode pads 104 may be adapted such that, in use, the electrode pads are placed at physiologically sensible locations on a user's body. For example, the electrode pads may be arranged such that the sensor may be ideally placed to measure a user's brain activity. Additionally or alternatively, the shape, size, number and placement of the electrode pads may be varied such that the sensor fits a variety of user groups or electrophysical monitoring functions. For example, the sensor, and accordingly the electrode pads may be smaller or larger to fit smaller (such as children) or large head sizes, or to have a different shape and size and/or number of electrode pads in order to serve as an electrode array for EKG, EEG or EMG, for example. The PCB 102 may take any suitable shape. The shape and design of the sensor, and therefore the placement of the electrode pads 104, may depend on where the sensor is to be used and the shape and size of this location. Maximizing user experience, i.e. reducing pain and discomfort, may be taken into consideration in the shape and design of the sensor.

FIG. 1B shows the sensor of FIG. 1A which is coated with an adhesive polymer composition. Thus, the present techniques provide an adhesive polymer composition comprising: a silicone polymer; and an amine-based polymer, wherein the adhesive polymer composition comprises from 1 to 10 wt. % amine-based polymer by weight of the adhesive polymer composition.

Advantageously, the adhesive polymer composition comprising a silicone polymer and an amine-based polymer (in the amount defined herein) means that the adhesive will adhere well to a user's skin. In particular, the adhesive polymer composition may be easily applied and removed from a user's skin. The adhesive polymer composition may be cleaned and reapplied several times without significantly losing its adhesive properties.

The silicone polymer and/or amine-based polymer may be capable of forming a substantially continuous film layer after curing.

The silicone polymer may comprise an elastomer and a curing agent. That is, the silicone polymer may be provided in two parts, and a curing process of the silicone polymer may only start when a curing agent is added to the silicone polymer base. It will be understood by a person skilled in the art that the silicone polymer, or polymer mixture, prior to the curing process will be uncured, and that after the curing process the silicone polymer may be crosslinked, suitably via a curing agent, to form a crosslinked polymer network in the final adhesive polymer composition. For the avoidance of doubt, by “uncured” is meant that the silicone polymer has not yet reacted with a curing agent (or otherwise) to form a crosslinked polymer network. The term “uncured adhesive composition” is herein used interchangeably with “adhesive polymer mixture” and “uncured adhesive polymer mixture”.

Advantageously, providing the silicone polymer and curing agent in two parts allows other components to be mixed with the elastomer base before a curing process starts, ensuring even distribution of any other components. The silicone polymer may preferably be inert and non-toxic, to ensure the conductive polymer composition is suitable for contact with human or animal skin. The silicone polymer may be any suitable material. The silicone polymer may be polydimethylsiloxane (PDMS). PDMS is an example of an elastomer, and it will be understood that any other suitable elastomers may be used, as long as they are also inert and non-toxic. Other non-limiting examples of silicone-containing polymers which may be particularly suitable for forming a film layer include polysiloxane polyureas, and polysiloxane polyamines.

The amine-based polymer may reduce or inhibit cross-linking bonds between the silicon base polymer and the curing agent for the silicone base polymer, creating heterogeneous cross-linking in the silicon polymer. The adhesive adheres to skin by van der Waals interactions.

In some cases, the silicone polymer may be polydimethylsiloxane.

In some cases, the amine-based polymer may be any of: polyethylenimine (PEI), including polyethylenimine PEI-derivatives such as ethoxylated polyethylenimine (PEIE), aminoethylaminopropyltrimethoxysilane (AEPS), and/or polyamidoamine (PAMAM). These may be used alone or in a mixture of two or more.

In some cases, the amine-based polymer may be polyethylenimine and/or ethoxylated polyethylenimine (PEIE).

In some cases, the amine-based polymer may be polyethylenimine.

In some cases, the amine-based polymer may be ethoxylated polyethylenimine (PEIE).

As noted above, the adhesive polymer composition comprises from 1 wt. % to 10 wt. % amine-based polymer by weight of the adhesive polymer composition. The adhesive polymer composition may comprise the amine-based polymer in a range from 1 wt. % to 2 wt. %, preferably in a range from 1.2 wt. % to 1.5 wt. %, more preferably in a range from 1.2 wt. % to 1.45 wt. %, more preferably in a range from 1.2 to 1.4 wt. %, most preferably in a range from 1.22 wt. % to 1.4 wt. %, by weight of the adhesive composition.

In some cases, the adhesive polymer composition may comprise the amine-based polymer in a range from 1.2 to 1.5 wt. % by weight of the adhesive composition.

In some cases, the adhesive polymer composition may comprise the amine-based polymer in a range from 1.2 to 1.4 wt. % by weight of the adhesive composition.

In some cases, the adhesive polymer composition may comprise from 1 wt. % to 10 wt. % polyethylenimine (PEI) by weight of the adhesive polymer composition. The adhesive polymer composition may comprise polyethylenimine (PEI) in a range from 1 wt. % to 2 wt. %, preferably in a range from 1.2 wt. % to 1.5 wt. %, more preferably in a range from 1.2 wt. % to 1.45 wt. %, more preferably in a range from 1.2 to 1.4 wt. %, most preferably in a range from 1.22 wt. % to 1.4 wt. %, by weight of the adhesive composition.

In some cases, the adhesive polymer composition may comprise polyethylenimine (PEI) in a range from 1.2 to 1.5 wt. % by weight of the adhesive composition.

In some cases, the adhesive polymer composition may comprise from 1 wt. % to 10 wt. % ethoxylated polyethylenimine (PEIE) by weight of the adhesive polymer composition. The adhesive polymer composition may comprise ethoxylated polyethylenimine (PEIE) in a range from 1 wt. % to 2 wt. %, preferably in a range from 1.2 wt. % to 1.5 wt. %, more preferably in a range from 1.2 wt. % to 1.45 wt. %, more preferably in a range from 1.2 to 1.4 wt. %, most preferably in a range from 1.22 wt. % to 1.4 wt. %, by weight of the adhesive composition.

In some cases, the adhesive polymer composition may comprise ethoxylated polyethylenimine (PEIE) in a range from 1.2 to 1.5 wt. % by weight of the adhesive composition.

In some cases, the sensor shown in FIG. 1B may also comprise flexible dry electrodes. The dry electrodes are also referred to herein as polymer electrodes or conductive polymer electrodes. The sensor 200 comprises at least one dry electrode 204 which may be provided on at least one electrode pad (not visible here as the electrode pads are below the dry electrodes 204, but see FIG. 1A) of the flexible printed circuit board. While the electrode pad of the F-PCB may be highly conductive, in order for the electrode pads to be used for surface electrophysiology (EEG, EMG or ECG for example) on the skin, a conductive interface between the F-PCB electrodes and the skin is necessary. This is because otherwise skin-electrode impedance is too high. In other words, it is very difficult/impossible to ensure that the electrode of the F-PCB makes full contact with the skin at all times without using a gel electrolyte interface. It is especially difficult to ensure sufficient contact when a user wearing the sensor moves. However, gel electrolyte interfaces are not desired, for the reasons explained above.

Thus, the sensor may further comprise at least one dry electrode 204 formed from an electrically-conductive polymer composition, the electrically-conductive polymer composition comprising: an electrically-conductive material; at least one additive; and a silicone polymer.

The conductive polymer composition is suitable for providing a dry electrode, i.e. an interface between an electrode pad 104 (which may be part of a sensor or wearable apparatus 100) and a user's skin. That is, the conductive polymer composition may reduce an electrode-skin impedance, such that an electrode/electrode pad 104 that is coated with the conductive polymer composition is able to better measure electrophysiological signals from a user's skin. Advantageously, an electrode 204 formed using the conductive polymer composition may be dry, thin (e.g. <1 mm in thickness) and flexible, meaning that the dry electrode 204 conforms to a user's skin without requiring a liquid or gel electrolyte interface. Thus, the conductive polymer composition may form a dry electrode 204 that leaves little or no residue on a user's skin, can be reapplied and cleaned several times and is comfortable for a user to wear.

As noted above, the conductive polymer composition comprises an electrically-conductive material. The electrically-conductive material is used in the conductive polymer composition to impart conductivity to the composition. The electrically-conductive material may be used without particular limitation as long as it has electron conductivity without causing chemical change. The electrically-conductive material may be a conductive allotrope of carbon, such as, for example, graphene, carbon nanotubes (CNTs), and graphite such as natural graphite and artificial graphite. The carbon may be doped or undoped. The carbon may be particulate carbon. The electrically-conductive material may be any of: carbonaceous materials such as carbon black, acetylene black, ketjen black, channel black, furnace black, lamp black, thermal black and carbon fiber; metal powder, metal fibers or metal nanoparticles such as copper, nickel, aluminum and silver; conductive whiskers such as zinc oxide and potassium titanate; conductive metal oxides such as titanium oxide; electrically-conductive polymers; conductive powders; and polyphenylene derivatives. These may be used alone or in a mixture of two or more.

In cases where the electrically-conductive material of the conductive polymer composition is provided by a carbon material, the carbon material may be a high conductivity carbon material. An example high conductivity carbon material is carbon black or particulate carbon black. However, it will be understood that this is a non-limiting example of a high conductivity carbon material.

The conductive polymer composition may comprise particulate carbon as the electrically-conductive material, and the particulate carbon may be in a range from 5 wt. % to 20 wt. % by weight of the electrically-conductive polymer composition. The particulate carbon may be present in a range from 12 wt. % to 18 wt. %, preferably in a range from 13 wt. % to 17 wt. %, more preferably in a range from 14 wt. % to 17 wt. %, by weight of the conductive polymer composition.

In some cases, the particulate carbon may be present in an amount of about 15 wt. % or 16 wt. % by weight of the conductive polymer composition.

In some cases, the particulate carbon may be present in an amount of about 14.9 wt. % or 15 wt. % by weight of the conductive polymer composition. The particulate carbon may be present in an amount of about 16 wt. % or 16.1 wt. % by weight of the conductive polymer composition.

The particulate carbon may be in any suitable form. In some cases, the particulate carbon may be in the form of nanoparticles. By “nanoparticles” is meant particles having an average diameter in the range of nanometers (nm). Particulate carbon nanoparticles may have an average diameter up to 900 nm, such as up to 800 nm, such as up to 700 nm, such as up to 600 nm, such as up to 500 nm, such as up to 400 nm, such as up to 300 nm, such as up to 200 nm, such as up to 100 nm. Particulate carbon nanoparticles may have an average diameter ranging from 5 to 500 nm, such as from 10 to 300 nm, such as from 20 to 250 nm, such as from 30 to 200 nm, such as from 40 to 150 nm, such as from 50 nm to 100 nm.

As noted above, the conductive polymer composition comprises at least one additive. The at least one additive may be, for example, a surfactant. The electrically-conductive polymer composition may comprise a surfactant to improve the miscibility of the composition, and/or to improve mechanical properties of the composition. The surfactant may be any suitable surfactant. For example, the surfactant may be an ionic or non-ionic surfactant. Preferably, the surfactant may be a non-ionic surfactant. The surfactant may be selected from any one of: those sold under the tradename Triton (commercially available from Dow), such as Triton X-100; those sold under the tradename Tween (commercially available from Croda), such as Tween 80, Tween 20, Tween 40, Tween 60 and/or mixtures thereof. These may be used alone or in a mixture of two or more. The surfactant may comprise Triton X-100 (also known as 2-[4-(2,4,4-trimethylpentan-2-yl)phenoxy]ethanol). The surfactant may comprise Tween 80 (also known as polyoxyethylene (20) sorbitan monooleate). However, it will be understood that these are non-limiting examples of a surfactant and that other surfactants may be used which have substantially similar properties.

In some cases, the at least one additive may be Triton X-100, and the Triton X-100 may be present in a range from 1 wt. % to 30 wt. %, preferably in a range from 1 wt. % to 20 wt. %, more preferably in a range from 5 wt. % to 15 wt. %, for example about 10 wt. % such as 10 wt. %, by weight of the conductive polymer composition.

In some cases, the at least one additive may be Tween 80, and the Tween 80 may be in a range from 1 wt. % to 30 wt. %, preferably in a range from 1 wt. % to 20 wt. %, more preferably in a range from 5 wt. % to 15 wt. %, for example about 10 wt. % such as 10 wt. %, by weight of the conductive polymer composition.

As noted above, the conductive polymer composition comprises a silicone polymer. The silicone polymer may comprise an elastomer base and a curing agent. That is, the silicone polymer may be provided in two parts, and a curing process of the silicone polymer may only start when a curing agent is added to the silicone polymer base. It will be understood by a person skilled in the art that the silicone polymer, or polymer mixture, prior to the curing process will be uncured, and that after the curing process the silicone polymer may be crosslinked, suitably via a curing agent, to form a crosslinked polymer network in the final adhesive polymer composition. For the avoidance of doubt, by “uncured” is meant that the silicone polymer has not yet reacted with a curing agent (or otherwise) to form a crosslinked polymer network. The term “uncured adhesive composition” is herein used interchangeably with “adhesive polymer mixture” and “uncured adhesive polymer mixture”.

Thus, the present techniques extend to a polymer mixture for forming an electrically-conductive polymer composition, the polymer mixture comprising an electrically-conductive material; at least one additive; a silicone polymer; and a curing agent.

Advantageously, providing the silicone polymer and curing agent in two parts allows other components to be mixed with the elastomer base before a curing process starts, ensuring even distribution of any other components. The silicone polymer may preferably be inert and non-toxic, to ensure the conductive polymer composition is suitable for contact with human or animal skin. The silicone polymer may be any suitable material. The silicone polymer may be polydimethylsiloxane (PDMS). PDMS is an example of an elastomer, and it will be understood that any other suitable elastomers may be used, as long as they are also inert and non-toxic.

On the proviso that a silicone polymer is present, other polymer materials may also be used without particular limitation as long as they have electron conductivity without causing chemical change. For example, non-limiting examples include polyurethanes.

The silicone polymer may comprise an elastomer base and a curing agent. That is, the silicone polymer may be provided in two parts, and a curing process of the silicone polymer may only start when a curing agent is added to the silicone polymer. Advantageously, this allows other components to be mixed before a curing process starts, ensuring even distribution of any other components.

As noted above, the conductive polymer composition comprises at least one additive. Thus, in some cases, the conductive polymer composition may comprise, at least, a first additive and a second additive. The first additive may be a surfactant, as noted above. The second additive may be a material which provides the conductive polymer composition with improved electrical conductivity properties. For example, the second additive may be ethylene glycol.

In cases where the second additive is ethylene glycol, the ethylene glycol may be in a range from 1 wt. % to 30 wt. %, preferably in a range from 1 wt. % to 20 wt. %, more preferably in a range from 3 wt. % to 15 wt. %, most preferably in a range from 6 wt. % to 8 wt. %, for example about 7 wt. % such as 7 wt. %, by weight of the conductive polymer composition.

As noted above, the electrically-conductive polymer composition may be used to improve the connectivity and conductivity between a skin-contacting component of a wearable sensor, and skin of a human or animal. Preferably, an electrical skin impedance of the conductive polymer composition (at 30 Hz) may be in a range from 100 kΩ to 2000 kΩ, preferably in a range from 100 kΩ to 1000 kΩ, more preferably in a range from 50 kΩ to 500 kΩ, most preferably in a range from 10 kΩ to 200 kΩ.

As shown in FIG. 1B, thin (e.g. <1 mm) films of the electrically-conductive polymer composition may be provided on top of the F-PCB electrode pads 104. The conductive polymer composition may cover part of or all of the electrode pad(s) 104 of the sensor 100.

Conductive polymers have favorable electrical (high conductivity) and physical (stretchability, flexibility, give) properties. Thus, using a conductive polymer composition to form a dry electrode allows the dry electrode to follow the surface of the skin very closely due to the flexibility, stretchability and “sponginess” of the dry electrode. This enables the dry electrode to achieve relatively low (<80-1000 kΩ) electrode-skin impedances without using electrolyte gel. Compared to any other type of dry electrode, the favorable physical properties of the conductive polymer electrode mean that there is far less variability in electrode-skin impedance from sensor to sensor. This has a great impact on an achievable signal to noise ratio (SNR) when measuring physiological signals using the dry electrodes. This is because minimizing variability means that the actual common mode rejection ratio achievable by a differential amplifier is maximized. The differential amplifier may be applied when receiving a signal measured by the sensor.

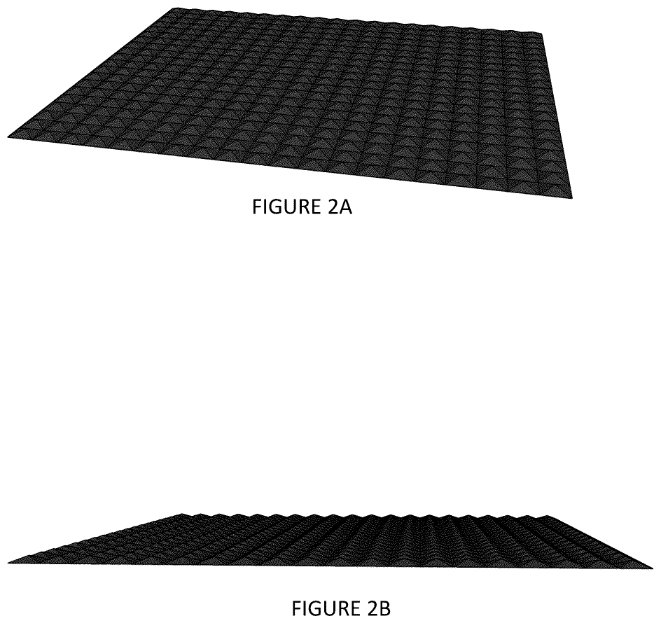

FIGS. 2A and 2B show an example pattern that may be applied to the dry electrodes of FIG. 1B. Thus, a surface of the dry electrode(s) may comprise patterning to obtain an increased surface area of the dry electrode. An increased surface area of the patterned dry electrode in turn ensures that a contact surface between the dry electrode and a user's skin is increased. Specifically, the effective area of contact between the electrode and the skin may be increased, which further lowers electrode-skin impedance. An increased contact surface improves the quality of an electrophysiological signal that can be measured by the at least one electrode pad 104 via the dry electrode 204 interface. Thus, an increase in surface area of the conductive polymer electrode improves a signal quality of the measured electrophysiological signal.

The pattern may be a 3D pattern of pyramids, as shown in FIGS. 2A and 2B, with 40 μm height and 200 μm centre-to-centre spacing. It will be understood that the pattern shown in FIGS. 2A and 2B is merely exemplary and non-limiting, and that any other suitable pattern may be used. For example, the pattern may be a 3D pattern of cubes, prisms or hemispheres. The individual elements of the pattern may vary in size and the sizes above are merely an example of suitable sizes. Other suitable sizes may be on the order of μm. In particular, the size of the individual elements of the pattern may be on the order of magnitude of the roughness of human skin, which is on the order of magnitude of dozens to hundreds of microns.

The pattern may, for example, be applied using a stamp. The stamp may, for example, be made from a polymer, such as polyurethane. For example, the stamp may be fabricated using a Polyuretane Ecoflex 00-30 kit by dispensing the same amount of Part A and Part B, 10 g of each, into a container (1A:1B by weight). Part A and Part B may then be mixed thoroughly for three minutes. Finally, the mixture may be poured it to a suitable box, for example, a plastic box. The mixture may be poured or spread out such that, after curing, the mixture forms an approximately 5 mm thick membrane. Curing may happen overnight at room temperature. After, the, for example, polyurethane membrane is cured, the membrane may be removed from the box. The pattern may be formed in the membrane using a laser, such as a CO₂ laser. As mentioned above, the pattern may be designed of a 2D pattern of squares with a 200×200 μm base area, resulting in a 3D pattern of pyramids with 60 μm height with 200 μm centre-to-centre spacing. The pattern may be made in Adobe Illustrator, for example, and applied using a Laser System VLS3.50: Cutting and Engraving Machine with the following parameters: power of 5%, speed of 100%, PPI of 1000. After forming the pattern using the laser, the membrane should be cleaned with Isopropyl alcohol (IPA) and Deionised (DI) water. Then, the stamp formed of the membrane may be placed on the uncured conductive polymer electrodes to apply the pattern to the conductive polymer electrodes. The pattern may be applied by applying pressure to the stamp by hand, or by any other suitable method. The stamp is then carefully removed from the conductive polymer electrodes. The resulting height of the pyramids is reduced in the conductive polymer electrode compared to the height of the pyramids in the stamp. For example, the conductive polymer electrode pattern may comprise pyramids of 40 μm height. The height of the pyramids may be in the range of skin roughness, which thereby helps the conductive polymer electrodes to conform to the skin.

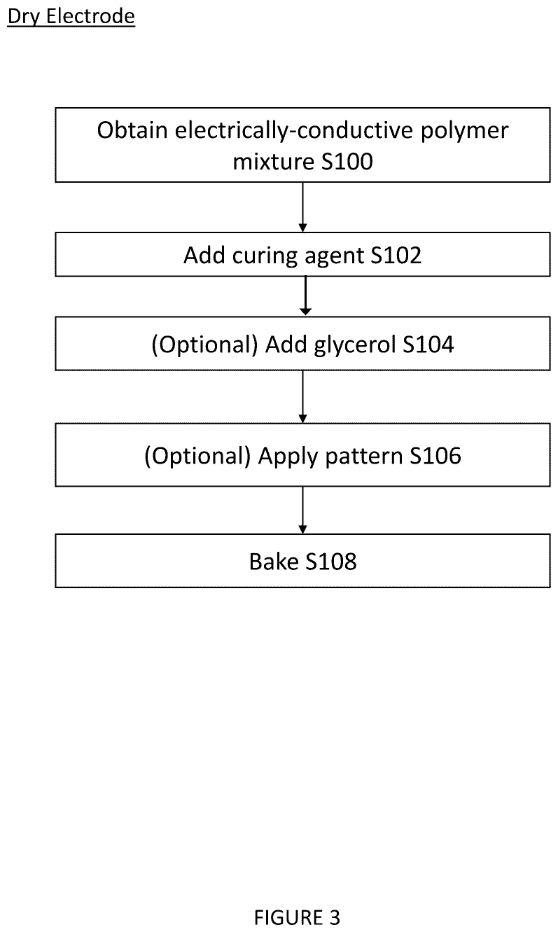

FIG. 3 is a flowchart showing the steps involved in producing the conductive polymer composition that may be used to form dry electrodes. The method comprises: obtaining an electrically-conductive polymer mixture (step S100). Obtaining, at step S100, the electrically-conductive polymer mixture may comprise: mixing an electrically-conductive material (e.g. one of the electrically-conductive materials described above), at least one additive (such as a surfactant or any of the additives described above), and a silicone polymer (or any other suitable silicone polymer-based material described above).

The method comprises adding a curing agent (step S102) to cure the electrically-conductive polymer mixture. That is, the silicone polymer may be provided in two parts, and a curing process of the silicone polymer may only start when a curing agent is added to the silicone polymer. Advantageously, this allows other components to be mixed with the elastomer before a curing process starts, ensuring even distribution of any other components.

The method then comprises thermally curing, such as by baking or otherwise heating, the polymer mixture (step S108). The cured electrically-conductive polymer may form a dry electrode on top of an electrode pad 104, for use in, for example, a wearable sensor 100.

A specific example of a dry electrode is now described, for illustrative purposes.

In this specific example, step S100 comprises mixing carbon, the silicone polymer base and at least one additive. For example, the following amounts of each substance may be added: 0.74 g (15 wt %) of carbon black, 3.3 g of PDMS part A, 0.35 g of ethylene glycol and 0.5 g of Triton X-100 and gently hand mixed for approximately five minutes, until all the carbon black is involved. Using 15w % of carbon black may achieve best conductivity of the conductive polymer. Using between 12-15 w % carbon black achieved good conductivity of the conductive polymer. That is 0.74 g of carbon black may be added to the above mixture. Alternatively, when adding 12w % of carbon black, 0.59 g (12 wt %) of carbon black may be added to the same amounts of other components.

Triton X-100 is a non-ionic surfactant that improves miscibility of carbon black and PDMS and thus improves the mechanical properties of the conductive polymer. That is, Triton X-100 acts as a ‘softener’, and depending on the amount of Triton X-100, this results in spongier or more solid electrodes.

Ethylene Glycol is a polar organic compound that works as a secondary dopant and thus improves the electrical properties of the conductive polymer.

Next, a curing agent may be added to the mixture at step S102. Using the quantities specified above, after obtaining a uniform ‘paste’, add 0.07 g of PDMS part B to the mixture and mix again for five minutes. PDMS part B is the curing agent for PDMS and is added only at the end to avoid the mixture curing during the fabrication process, ensuring a better uniformity of the final paste.

The silicone polymer may be polydimethylsiloxane which is created when PDMS Part A (silicone polymer base) and Part B (curing agent) react and cure. When heated, PDMS Part A and Part B crosslink, forming a network structure of chemical bonds. That is, heat curing results in the formation of a silyl hybrid group due to contact between a vinyl or allyl group and a silicone-based material.

Optionally, as shown in FIG. 3, glycerol may be added at this stage (step S104). Adding glycerol as a further additive may improve the adhesive properties of the conductive polymer electrode. For example, 0.55 g of glycerol may be added to the mixture with quantities specified above at this stage if using 14-15 wt % of carbon black or 1 g of glycerol if using 12-14 wt % of carbon black. Alternatively, when adding glycerol, 0.66 g may be added to the composition, with the above specified quantities of the other components.

Thus, to summarize, the following components may be used to obtain the example electrically-conductive polymer electrode composition described above:
Carbon black (C), C-Nergy Super C65, ≥99.975, PI-KEM
Ethylene Glycol—RPE (CH₂OHCH₂OH), 99.5% (GLC), Carlo Erba Reagents
Triton X-100 ((C₂H₄O)ₙC₁₄H₂₂O), alkylaryl polyether alcohol, ≤100%, BAKER ANALYZED, J. T. Baker®
Polydimethylsiloxane ((C₂H₆OSi)ₙ), Sylgard 184 Silicone Elastomer Kit, Dow:
Part A—Sylgard 184 Silicone Elastomer Base:
Dimethyl siloxane, dimethylivinyl terminated—68083-19-2
Dimethylvinylated and trimethylated silica—68988-89-6
Tetra (trimethoxysiloxy) silane—3555-47-3
Ethyl benzene—100-41-4
Part B—Sylgard 184 Silicone Elastomer Curing Agent:
Dimethyl, methylhydrogen siloxane—68037-59-2
Dimethyl siloxane, dimethylvinyl terminated—68083-19-2
Dimethylvinylated and trimethylated silica—68988-89-6
Tetramethyl tetravinyl cyclotetra siloxane—2554-06-5
Ethyl benzene—100-41-4
Glycerol (HOCH₂CH(OH)CH₂OH), BioXtra, >=99% (GC), Sigma Aldrich

The components of the conductive polymer may be present in the conductive polymer in the following proportions:

| Component | Mass (g) | w/w (solute/solution) % |
|---|---|---|
| PDMS part A (base) | 3.3 | 66.532 |
| PDMS part B (curing agent) | 0.07 | 1.411 |
| Carbon black | 0.74 | 14.919 |
| Ethylene Glycol | 0.35 | 7.056 |
| Triton X-100 | 0.5 | 10.081 |

The above amounts refer to both the cured and uncured conductive polymer. Merely for illustrative purposes, the above table includes wt. %, as well as an example of a mixed composition given in units of mass (g). Different proportions of the constituent parts of the conductive polymer composition are possible. The conductive polymer composition may comprise at least 5 wt % particulate carbon. The conductive polymer composition may comprise no more than 20 wt % particulate carbon. The conductive polymer composition may comprise at least 9w % particulate carbon. The conductive polymer composition may comprise no more than 16w % particulate carbon. Particulate carbon may mean carbon black, as described above. The conductive polymer composition may comprise 14.9 w % particulate carbon. The lower limit of carbon black is dictated by conductivity requirements, whereas the higher limit is dictated by mechanical properties of the silicone polymer as well as the curing process. Adding more than 20 wt %, or in some cases more than 16 wt %, of carbon black may result in less favorable mechanical properties of the cured conductive polymer and higher skin impedance. However, in order to reach conductivity that is as high as possible, it may be advantageous to include enough carbon black as possible, without affecting the mechanical properties of the conductive polymer.

The conductive polymer composition may comprise 10 w % Triton X-100. The conductive polymer composition may comprise 7w % ethylene glycol. The resulting conductive polymer composition may have an electrical skin impedance of less than 2000 kΩ. The electrical skin impedance of the conductive polymer may be between 80 kΩ and 1000 kΩ.

Returning to FIG. 3, the method may comprise patterning a surface of the film of conductive polymer composition prior to curing (step S106). Preferably, this step is performed after the film of the conductive polymer composition has been applied to the at least one electrode pad 104 of the F-PCB. Thus, manufacturing a conductive polymer electrode may further comprise patterning a surface of the uncured conductive polymer to obtain an increased surface area of the cured conductive polymer/dry electrode 204.

Curing the conductive polymer mixture at step S108 may comprise at least one thermal curation step at a predetermined curation temperature. Controlling a temperature of a thermal curation step ensures that the curation process is controlled and therefore, along with controlling a ratio of a curing agent in the conductive polymer, allows control over the ratio of cured to uncured polymer chains in the cured conductive polymer. Controlling a temperature of a thermal curation step ensures that the curation process is controlled and therefore allows control over the ratio of cured to uncured polymer chains in the cured conductive polymer. In turn, control over the ratio of cured to uncured polymer chains means that the properties of the conductive polymer can be controlled. For example, a larger number of cured chains results in a more solid conductive polymer, whereas less cured chains result in a softer conductive polymer. These are important properties, as the softness of the conductive polymer and other surface properties of the conductive polymer determine how well the conductive polymer conforms to a user's skin and thus how well the conductive polymer works as an interface between the electrode pad and the user's skin.

Thus, the sensor, with the at least one dry electrode, may be placed in an oven at step S108. The temperature of the oven may be in a range from room temperature to 110° C. The oven may be programmed to gradually increase the temperature from room temperature to a predetermined temperature of 100° C. over a predetermined time period of two hours. Next, the temperature may be in a range from 100° C. to 110° C. The oven may be programmed to increase the temperature from 100° C. to 110° C. over a two-hour period. That is, there may be a further increase in temperature of 5° C. per hour. The second rise in temperature is a slow rise in temperature, to avoid any cracking or other temperature-related defects on the conductive polymer electrodes. Finally, the temperature may decrease from 110° C. to 24° C. Thus, during this final step, the temperature may be lowered from 110° C. to room temperature over a 2.5 hour period.

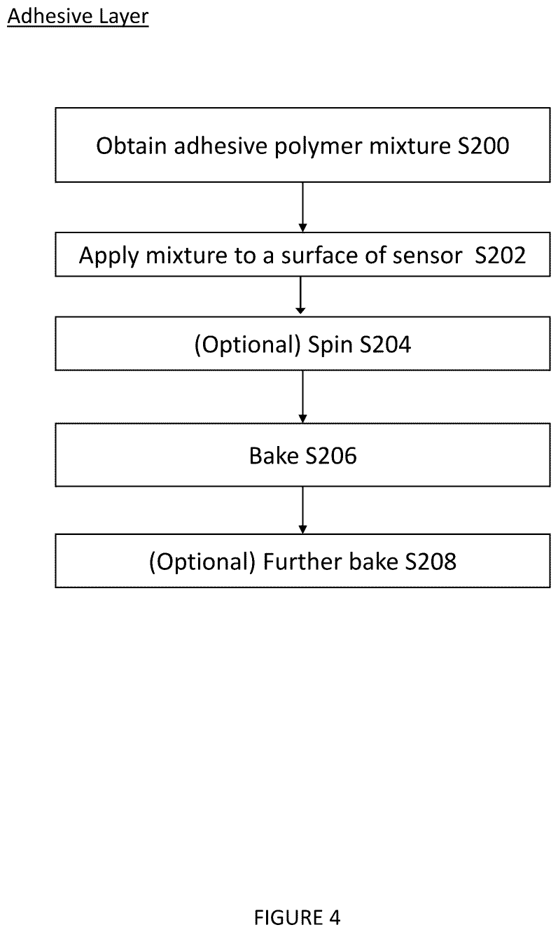

FIG. 4 is a flowchart showing the steps involved in manufacturing a sensor having an adhesive polymer layer. As shown in FIG. 1B, the sensor 200, and in particular the flexible substrate of the sensor, may be coated with an adhesive 202 which is applied to at least part of the sensor surface on which there are no electrodes. That is, the area of the sensor around the electrodes may, at least partially, be coated with an adhesive 202.

In cases where the sensor comprises both an adhesive polymer coating/layer and dry electrodes, the steps of FIG. 3 may be performed before the steps of FIG. 4, or some steps of FIG. 3 may be performed at substantially the same time as some of the steps of FIG. 4.

The method to form the adhesive coating or adhesive layer on the flexible substrate comprises: obtaining an adhesive polymer mixture (step S200). The adhesive may comprise at least one opening that corresponds to a position of the at least one electrode pad. Coating the flexible substrate in an adhesive ensures an even and comfortable fit of the sensor for a user. Advantageously, an even and skin conformal fit ensures that no motion artefacts are introduced to an electrophysiological signal measured by the electrode pad by movement of a user. Additionally, coating the flexible substrate in an adhesive can ensure that application of the sensor is straightforward and user-friendly.

When adhesive is placed around the conductive polymer electrodes on the sensor, advantageously, an adhesive layer may have the same or a similar thickness as the conductive polymer electrodes. That is, the surface of the sensor, formed by the adhesive and the at least one conductive polymer electrode may be thin (<1 mm). The adhesive may be of similar thickness as the conductive polymer electrode coating that is applied to the at least one electrode pad. This ensures that the sensor fits evenly to a user's skin and ensures that the fit of the sensor is skin conformal. However, there may be a difference in thickness between the adhesive and the conductive polymer. The conductive polymer may be slightly raised on the surface of the sensor over the applied adhesive. This difference in thickness between the conductive polymer and the adhesive may be in a range from 0 μm to 300 μm. Preferably, the difference in thickness may be in a range from 50 μm to 200 μm. More preferably, in a range from 100 μm to 150 μm, for example about 125 μm such as 125 μm. When the conductive polymer layer is slightly thicker than the adhesive layer, the adhesive pushes the conductive polymer layer against a user's skin, improving contact between the electrode and the user's skin. As described above, the surface of the conductive polymer electrodes may comprise some patterning.

Obtaining, at step S200, an adhesive polymer composition comprises obtaining an adhesive polymer mixture by mixing a silicone polymer and an amine-based polymer.

The silicone polymer and/or amine-based polymer may be capable of forming a substantially continuous film layer after curing.

The silicone polymer may comprise an elastomer and a curing agent. That is, the silicone polymer may be provided in two parts, and a curing process of the silicone polymer may only start when a curing agent is added to the silicone polymer base. It will be understood by a person skilled in the art that the silicone polymer, or polymer mixture, prior to the curing process will be uncured, and that after the curing process the silicone polymer may be crosslinked, suitably via a curing agent, to form a crosslinked polymer network in the final adhesive polymer composition. For the avoidance of doubt, by “uncured” is meant that the silicone polymer has not yet reacted with a curing agent (or otherwise) to form a crosslinked polymer network. The term “uncured adhesive composition” is herein used interchangeably with “adhesive polymer mixture” and “uncured adhesive polymer mixture”.

Advantageously, providing the silicone polymer and curing agent in two parts allows other components to be mixed with the elastomer base before a curing process starts, ensuring even distribution of any other components. The silicone polymer may preferably be inert and non-toxic, to ensure the conductive polymer composition is suitable for contact with human or animal skin. The silicone polymer may be any suitable material. The silicone polymer may be polydimethylsiloxane (PDMS). PDMS is an example of an elastomer, and it will be understood that any other suitable elastomers may be used, as long as they are also inert and non-toxic. Other non-limiting examples of silicone-containing polymers which may be particularly suitable for forming a film layer include polysiloxane polyureas, and polysiloxane polyamines.

The amine-based polymer may reduce or inhibit cross-linking bonds between the silicon base polymer and the curing agent for the silicone base polymer, creating heterogeneous cross-linking in the silicon polymer. The adhesive adheres to skin by van der Waals interactions.

In some cases, the silicone polymer may be polydimethylsiloxane.

The amine-based polymer may be any suitable material. For example, the amine-based polymer may be any of: polyethylenimine (PEI), including polyethylenimine PEI-derivatives such as ethoxylated polyethylenimine (PEIE), aminoethylaminopropyltrimethoxysilane (AEPS), and/or polyamidoamine (PAMAM). These may be used alone or in a mixture of two or more.

In some cases, the amine-based polymer may be polyethylenimine and/or ethoxylated polyethylenimine (PEIE).

In some cases, the amine-based polymer may be polyethylenimine.

In some cases, the amine-based polymer may be ethoxylated polyethylenimine (PEIE).

As noted above, the adhesive polymer composition comprises from 1 wt. % to 10 wt. % amine-based polymer by weight of the adhesive polymer composition. In some cases, the adhesive polymer composition may comprise the amine-based polymer in a range from 1 wt. % to 2 wt. %, preferably in a range from 1.2 wt. % to 1.5 wt. %, more preferably in a range from 1.2 wt. % to 1.45 wt. %, more preferably in a range from 1.2 to 1.4 wt. %, most preferably in a range from 1.22 wt. % to 1.4 wt. %, by weight of the adhesive composition.

In some cases, the adhesive polymer composition may comprise the amine-based polymer in a range from 1.2 to 1.5 wt. % by weight of the adhesive composition.

In some cases, the adhesive polymer composition may comprise the amine-based polymer in a range from 1.2 to 1.4 wt. % by weight of the adhesive composition.

In some cases, the adhesive polymer composition may comprise from 1 wt. % to 10 wt. % polyethylenimine (PEI) by weight of the adhesive polymer composition. In some cases, the adhesive polymer composition may comprise polyethylenimine (PEI) in a range from 1 wt. % to 2 wt. %, preferably in a range from 1.2 wt. % to 1.5 wt. %, more preferably in a range from 1.2 wt. % to 1.45 wt. %, more preferably in a range from 1.2 to 1.4 wt. %, most preferably in a range from 1.22 wt. % to 1.4 wt. %, by weight of the adhesive composition.

In some cases, the adhesive polymer composition may comprise polyethylenimine (PEI) in a range from 1.2 to 1.5 wt. % by weight of the adhesive composition.

As such, in some cases, the adhesive polymer composition may comprise from 1 wt. % to 10 wt. % ethoxylated polyethylenimine (PEIE) by weight of the adhesive polymer composition. The adhesive polymer composition may comprise ethoxylated polyethylenimine (PEIE) in a range from 1 wt. % to 2 wt. %, preferably in a range from 1.2 wt. % to 1.5 wt. %, more preferably in a range from 1.2 wt. % to 1.45 wt. %, more preferably in a range from 1.2 to 1.4 wt. %, most preferably in a range from 1.22 wt. % to 1.4 wt. %, by weight of the adhesive composition.

In some cases, the adhesive polymer composition may comprise ethoxylated polyethylenimine (PEIE) in a range from 1.2 to 1.5 wt. % by weight of the adhesive composition.

The silicone polymer may be polydimethylsiloxane which is caused to crosslink when PDMS Part A (silicone polymer base) and Part B (curing agent) react and cure. When heated, PDMS Part A and Part B crosslink, forming a network structure of chemical bonds.

Advantageously, the adhesive is skin-friendly, removable without or with only little residue and ensures that a fit of the conductive polymer electrodes to the user's skin is skin-conformal. Additionally, the adhesion provided by the adhesive may be renewable—that is the sensor may be reapplied several times. For example, the adhesion may be renewable over a one-week period, and thus a user can reuse the sensor over a prolonged period of time. To renew the adhesion, the sensor and the adhesive applied to the sensor may be rinsed with (70%) ethanol or any other suitable alcohol-based solution (such as isopropyl alcohol). Rinsing the sensor with ethanol removes the dust (micro-particles) on the surface of the adhesive. Micro-particles are responsible for reducing the area of contact and van der Walls interactions with the human skin. This ethanol rinsing step allows the adhesive of the sensor to retain adhesive properties. Other bonding mechanisms may be possible. That is, alternative mechanisms of the adhesive bonding to the user's skin may be used, and thus the adhesive may comprise any suitable adhesive polymer, such as any suitable amine-based polymer or any suitable non-amine-based polymer.

For example, the following components may be used to obtain the adhesive silicone and amine-based polymer composition described above:
Polydimethylsiloxane ((C₂H₆OSi)ₙ), Sylgard 184 Silicone Elastomer Kit, Dow:
Part A—Sylgard 184 Silicone Elastomer Base:
Dimethyl siloxane, dimethylivinyl terminated—68083-19-2
Dimethylvinylated and trimethylated silica—68988-89-6
Tetra (trimethoxysiloxy) silane—3555-47-3
Ethyl benzene—100-41-4
Part B—Sylgard 184 Silicone Elastomer Curing Agent:
Dimethyl, methylhydrogen siloxane—68037-59-2
Dimethyl siloxane, dimethylvinyl terminated—68083-19-2
Dimethylvinylated and trimethylated silica—68988-89-6
Tetramethyl tetravinyl cyclotetra siloxane—2554-06-5
Ethyl benzene—100-41-4
Polyethylenimine (PEIE), 80% ethoxylated solution

The components of the adhesive polymer may be present in the adhesive polymer in the following proportions:

| Component | Mass (g) | w/w (solute/solution) % |
|---|---|---|
| PDMS part A (base) | 100 | 93.985 |
| PDMS part B (curing agent) | 5 | 4.699 |
| PEIE | 1.35-1.45 | 1.269-1.362 |

The above amounts refer to both the cured and uncured adhesive polymer composition. Outside of the w/w (solute/solution) percentages range, the adhesive may not cure.

Returning to FIG. 4, step 200 comprises mixing the amine-based polymer, curing agent and silicone base. Mixing may take place by hand for about three minutes. The amine-based polymer may be Polyethylenimine (PEIE). The silicone base may be Part A—Sylgard 184 Silicone Elastomer Base (PDMS) and the curing agent may be Part B—Sylgard 184 Silicone Elastomer Curing Agent (a curing agent for PDMS). The silicone elastomer may hence also be referred to as PDMS Part A, and the curing agent may be referred to as PDMS Part B. Mixing the components of the adhesive polymer composition may comprise mixing 1.4 g of PEIE, 5 g of PDMS Part B and 100 g of PDMS Part A. When mixing the components of the adhesive polymer, PEIE and PDMS Part B may be mixed first, with PDMS Part A added in the last step of the mixing process. Alternatively, mixing the components of the adhesive polymer may comprise mixing 2.52 g of PEIE, 9 g of PDMS Part B and 171 g of PDMS Part A.

Next, the adhesive polymer may be deposited on the sensor (step S202). Deposition on the sensor may happen on the whole surface area of the sensor that comprises the electrode pads of the sensor. Alternatively, deposition may take place on part of the surface area of the sensor only. For example, the adhesive polymer may be deposited around the edges of the sensor only, or around the electrode pads of the sensor only. When the adhesive polymer is deposited on the whole surface area of the sensor, the adhesive polymer may cover the electrode pads of the F-PCB of the sensor. Subsequently, the adhesive polymer covering the electrode pads may then be removed. Removal of adhesive polymer covering the electrode pads may take place after the sensor that is coated in the adhesive polymer is baked (step S206). This is because it may be easier to cut out holes for applying the conductive polymer electrode composition after the adhesive has undergone a curing process, by baking for example, during which at least part of the adhesive becomes a solid. Curing the adhesive polymer composition may comprise inducing a thermal curation process in the adhesive, for example by exposing the adhesive polymer composition to heat. Additionally or alternatively, curing the adhesive polymer composition may lead to the adhesive polymer composition becoming at least partially desiccated. When removing adhesive polymer covering the electrode pads after baking, the adhesive polymer may be removed by cutting/burning the adhesive polymer away with a laser, such as, for example, a CO₂ laser. Additionally, or alternatively, the adhesive may be removed by any other suitable method. For example, the adhesive may be cut and/or scraped away. The adhesive may be burnt away using other suitable methods. The adhesive may be removed using a solvent. Any of the previously mentioned removal methods may be combined. For example, the adhesive may first be cut/burned away using a laser and any remaining adhesive residue may then be cut/scraped away using a scalpel or other suitable tool.

Optionally, before curing at step S206 the adhesive polymer composition, after the adhesive polymer composition has been deposited at step S202 on the sensor, the sensor with the adhesive polymer coating may be spun (step S204), to ensure an even distribution of the adhesive polymer on the sensor. That is, the sensor may be spin-coated with the adhesive polymer. For example, the following spin-coating conditions may be used: (1-5s, 1000 rpm, 1000 acc). These spin-coating conditions result in a thin film coating of the adhesive polymer with an average thickness of around 300 μm. Other suitable methods for distributing the adhesive polymer coating on the sensor may be used. Other suitable spin-coating conditions or other suitable coating methods or patterning methods may be used to create a coating of the adhesive polymer on the sensor. The thickness of the adhesive polymer coating may vary. For example, the thickness of the adhesive polymer coating may be anywhere between 100-650 μm.

Curing the adhesive polymer composition at step S206 may comprise placing the sensor with the adhesive polymer composition in an oven. Curing may comprise baking the adhesive polymer and curing the adhesive polymer mixture may comprise at least one thermal curation step. The at least one thermal curation step may take place at a predetermined curation temperature over a predetermined curation period. Controlling a temperature of a thermal curation step ensures that the curation process is controlled and therefore, along with controlling a ratio of a curing agent in the polymer, allows control over the ratio of cured to uncured polymer chains in the cured adhesive polymer. In turn, control over the ratio of cured to uncured polymer chains means that the properties of the adhesive polymer can be controlled. For example, a larger number of cured chains results in a more solid and less adhesive polymer, whereas less cured chains result in a softer and more adhesive polymer. These are important properties, as the softness and adhesiveness of the adhesive polymer determine how well the adhesive polymer conforms to a user's skin and thus how well the sensor as a whole conforms to a user's skin and consequently functions. The at least one thermal curation step may comprise at least two thermal curation steps. A sequence of thermal curation steps may help ensure that the cured adhesive polymer has the required properties. The at least two thermal curation steps may comprise a first thermal curation step taking place at a first predetermined curation temperature over a first predetermined curation period, and a second thermal curation step taking place at a second predetermined curation temperature over a second predetermined curation period. The at least one thermal curation step may comprise at least three thermal curation steps. The at least three thermal curation steps may comprise a first thermal curation step taking place at a first predetermined curation temperature over a first predetermined curation period, and a second thermal curation step taking place at a second predetermined curation temperature over a second predetermined curation period, and a third thermal curation step taking place at a third predetermined curation temperature over a third predetermined curation period.

In the first thermal curation step, a first curation temperature may be in a range from room temperature to 100° C. For example, the oven may be programmed to gradually increase the temperature from room temperature to 100° C. over a first predetermined curation period of two hours. Next, a second curation temperature may be in a range from 100° C. to 130° C. That is, the temperature may further be increased to 130° C. over a second predetermined curation period of two hours. That is, there may be a further increase in temperature of 15° C. per hour. The sensor coated with the adhesive polymer may be baked at a third curation temperature which may be in a range from 110° C. to 150° C. Preferably, the third curation temperature may be in a range from 120° C. to 140° C. More preferably, the third curation temperature may be in a range from 125° C. to 135° C. For example, the third curation temperature may be 130° C. The sensor coated with the adhesive polymer may thus be baked for a further four hours in a third thermal curation step. Finally, the oven may be turned off and left to cool, until the temperature returns to room temperature, during which time the adhesive polymer coating stabilizes.

Optionally, in cases where the sensor comprises dry electrodes and an adhesive, when the adhesive polymer is applied before the conductive polymer electrode is applied to the sensor, the adhesive polymer may be further baked at step S208 with the conductive polymer electrode. That is, there may be at least one further baking step, with the baking temperature and time of the bake adjusted such that the conductive polymer electrode is cured optimally. The baking process of step S208 may thus comprise identical temperatures and timings to that of S108 of FIG. 3 described above.

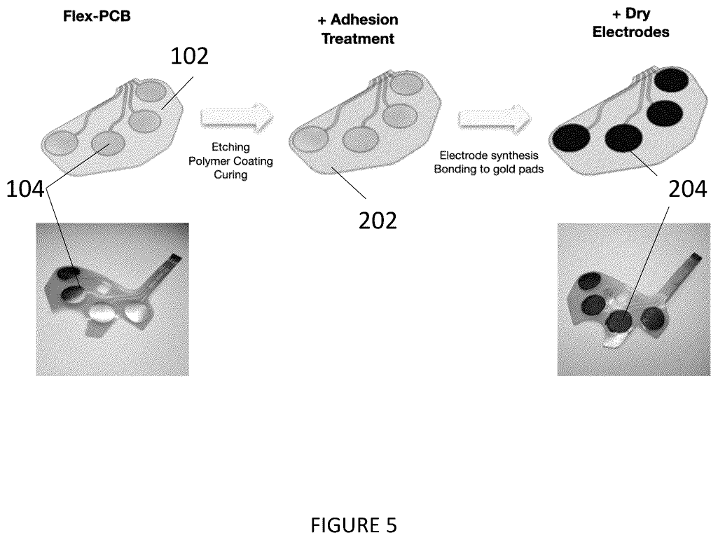

FIG. 5 is a diagram showing an overview of the overall process for coating the F-PCB of the sensor with an adhesive and the conductive polymer electrode composition. As shown in FIG. 5, and explained above, the present techniques provide a wearable sensor for monitoring physiological and/or brain signals, the sensor comprising: a thin flexible substrate comprising a skin-contacting surface, wherein the skin-contacting surface is at least partially coated in the adhesive composition described herein; and at least one electrode pad for monitoring physiological and/or brain signals, wherein the at least one electrode pad is at least partially coated in a coating formed of a conductive polymer composition. The conductive polymer composition coating may form a dry electrode 204 over each electrode pad 104, as shown in FIG. 5.

The coating may comprise patterning, as described above with reference to FIG. 2, to obtain an increased surface area of the conductive polymer. This may improve the ability for the coating (i.e. dry electrode) to contact the skin and sense signals.

The at least one electrode pad (and the coating) and the circuitry may be provided on a thin flexible substrate, and the circuitry may also be flexible. The substrate may have a thickness in a range from 0.04 to 0.3 mm. For example, the substrate may have a thickness of 0.08 mm. The substrate may be made from flexible materials, such as, for example, a polymer and/or polyimide layer and/or a conductive material, such as copper with a thickness in a range from 10 to 25 μm, for example, copper with a thickness of 18 μm. At least part of the substrate may have an immersion gold surface finish. A thin flexible substrate and flexible circuitry mean that the sensor adapts to a user's movement—the sensor moves along with the user's skin, even when the user is moving. Advantageously, this means that contact between a conductive polymer electrode interface and the user's skin is always maintained. Additionally, with the circuitry being flexible and provided on the flexible substrate, the circuitry will also move along with any movements of the user. The flexible, skin-conformal nature of the at least one electrode allows it to follow and make full contact with the surface of the skin, even as it deforms during movement, enhancing the signal quality obtained. The adhesive polymer coating enables the at least one electrode to stick to and remain in place relative to the skin, as the user moves, preventing any relative displacement between the sensor and the skin. The combination of these two elements directly attacks the cause of motion artefacts and eliminates them. Thus, a thin conductive polymer electrode, a flexible substrate and flexible circuitry improve a signal quality of an electrophysiological signal measured by the electrode pads.

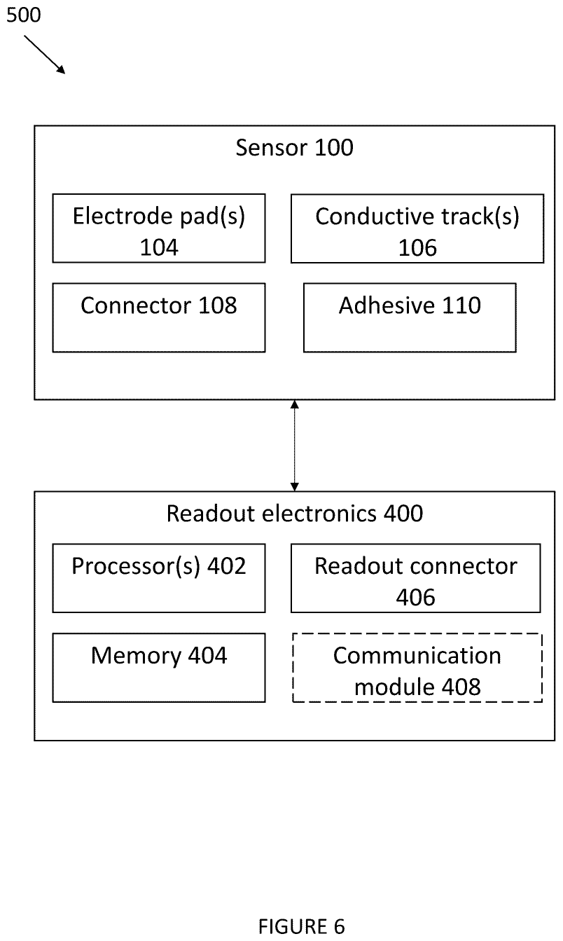

FIG. 6 is a schematic block diagram of a system for measuring physiological and/or brain signals. The system 500 may comprise the sensor 100 as described above and a readout electronics 400 device.

The sensor 100 comprises an adhesive 110, provided by the adhesive polymer composition described herein. The adhesive 110 may be in the form of a layer on a skin-contacting surface of the sensor 100, as explained above.

The sensor 100 comprises at least one electrode pad 104. The at least one electrode pad 104 may be used for EEG, ECG and/or EMG measurements (which are the measurements most susceptible to motion-induced noise) or measurement of any other surface electrophysiological signal from the body. When the sensor is provided on a user's head, the at least one electrode pad may sense brain signals. In this case, the at least one electrode pad may be able to provide (directly or indirectly) information on the cognition, emotional state, or disease indicators. The sensor may further comprise at least one conductive track 106. The at least one conductive track 106 may transmit measurements from the at least one electrode pad to a connector 108. The sensor 100 may also comprise the connector 108 onto the PCB 102. The connector may be used to connect the sensor 100 to an apparatus and/or readout electronics for receiving a physiological signal that is measured by the sensor 100.

The system may further comprise readout electronics 400. The readout electronics may be connected via a readout connector 406 to the sensor's connector 108. The readout electronics may further comprise at least one processor 402 and memory 404. The processor and memory may be used to analyze physiological and/or brain signals measured by the sensor. Optionally, the readout electronics may also comprise a communication module 408 for transmitting a received physiological and/or brain signal to an external electronic device. Thus, the readout electronics may wirelessly transmit data to a mobile, desktop or cloud computing platform for further data processing and extraction of insights relating to a cognitive and physiological state of a user.

The readout electronics 400 may comprise: an analog front-end (which comprises an amplifier and a digitizer), a micro-controller unit, a wireless communication module (e.g. cellular, WiFi, Bluetooth, etc.) and other passive circuit elements required to make these components work.

### Example 1

An adhesive polymer composition in accordance with the present invention was prepared according to the following table:

| Component | Mass (g) | w/w (solute/solution) % |
|---|---|---|
| Sylgard 184 Silicone Elastomer * | 100 | 94.0 |
| (PDMS part A; base) | | |
| Sylgard 184 Silicone Elastomer | 5 | 4.7 |
| Curing Agent * (PDMS part B; | | |
| curing agent) | | |
| PEIE, 80% ethoxylated solution | 1.4 | 1.3 |

* Commercially available from Dow as a two-part kit

The PEIE and PDMS Part B were mixed first, before PDMS Part A was added and mixed in the last step of the mixing process. Next, the adhesive polymer mixture was deposited on an electrode pad or a wearable sensor and cured in an oven using the following curing process:
1^{st} step: room temperature to 100° C. over two hours;
2^{nd} step: 100° C. to 130° C. over two hours;
3^{rd} step: 130° C. for four hours;

The oven was then turned off and left to cool until the temperature returns to room temperature.

The adhesion of the curing adhesive polymer composition was assessed by the tensile strength test, whereby the maximum tension force that the adhesive polymer composition can withstand when adhered to a metal substrate and subsequently peeled off from said substrate was recorded. The testing was performed using a Minimat Mini-Mechanical Testing machine (Load cell: 20 N; Maximum displacement: 90 cm). The results shown below are an average.

Results are shown below.

### Comparative Example 1

The comparative example is as shown and tested in Jeong et al, PDMS-based Elastomer Tuned Soft, Stretchable, and Sticky for epidermal electronics, Advanced Materials, 2018, 28, 5830-5836 (DOI: 10.1002/adma.201505372; ‘Jeong et al’). As shown in Table 1 of Jeong et al, and as described in the accompanying Experimental Section starting on page 5834, a PDMS-based elastomer (Sylgard184) was prepared by adding 40 μL PEIE (80% solution of ethoxylated polyethylenimine) to 10 g of the PDMS-based elastomer. The composition therefore had the following components.

| Component | Mass (g) | w/w (solute/solution) % |
|---|---|---|
| Sylgard 184 Silicone Elastomer * | 9 | 89.6 |
| (PDMS part A; base) | | |
| Sylgard 184 Silicone Elastomer | 1 | 10.0 |
| Curing Agent * (PDMS part B; | | |
| curing agent) | | |
| PEIE, 80% ethoxylated solution | 0.04 | 0.4 |

The mixture was cured at 90° C. for 2-3 hours in an oven.

The comparative composition was tested according to adhesion force testing as follows: The adhesion test of the S3-PDMS was done with a dynamometer (no. 22, 200 g, MWT) and a linear guide stage (A-LSQ, Zaber). The dynamometer (a spring force gauge) was fixed to the linear guide and the test sample was attached on the forearm skin that was clamped with one end of the sample by a plastic clip. The sample size was 25 mm wide and 70 mm long. For the adhesion force test sample preparation, the mixture of the S3-PDMS was laminated on a plastic film (Transparency film, Canon) with a film applicator (PA2041, BYK). The thickness of the laminated layers was 50 μm. The peeling speed was controlled by the linear guide and the peeling speed was 20 mm s⁻¹ with 100 mm stroke. The maximum adhesion force was read during peeling off the test sample from the skin of the forearm.

Results are shown below.

Results

| | Adhesion | | Adhesion Force |
|---|---|---|---|
| | Force(N) | Area (cm²) | (N/cm²) |
| Comparative | 1.2 | 17.5 | 1.2 |
| Example 1 | | (2.5 × 7) | |
| Example 1 | 8 | 2 | 4 |
| | | (1 × 2) | |

For the intended application the adhesive polymer needs a certain level of adhesion, preferably at least 1.5 N/cm², more preferably at least 2 N/cm², even more preferably at least 2.5 N/cm², and most preferably at least 3 N/cm². The results show that the use of PEIE in an amount ≥1 wt. % (1.4 wt. %) enables an adhesion force around 4 N/cm² (8 N for a sample of 2×1 cm²). In comparison, the use of ≤1 wt. % (0.4 wt. %) results in poor adhesion (<1.5 N/cm²).

Those skilled in the art will appreciate that while the foregoing has described what is considered to be the best mode and where appropriate other modes of performing present techniques, the present techniques should not be limited to the specific configurations and methods disclosed in this description of the preferred embodiment. Those skilled in the art will recognize that present techniques have a broad range of applications, and that the embodiments may take a wide range of modifications without departing from any inventive concept as defined in the appended claims.

The invention may be defined in accordance with the following numbered aspects:
1. A wearable sensor for monitoring physiological and/or brain signals, the sensor comprising: a thin flexible substrate comprising a skin-contacting surface, wherein the skin-contacting surface is at least partially coated in an adhesive polymer composition, the adhesive polymer composition comprising: a silicone polymer; and an amine-based polymer, wherein the adhesive polymer composition comprises from 1 to 10 wt. % amine-based polymer by weight of the adhesive polymer composition.
2. The wearable sensor according to aspect 1 wherein the silicone polymer is polydimethylsiloxane.
3. The wearable sensor according to aspect 1 or 2 wherein the amine-based polymer is selected from any one of polyethylenimine, including polyethylenimine PEI-derivatives such as ethoxylated polyethylenimine (PEIE), aminoethylaminopropyltrimethoxysilane (AEPS), and/or polyamidoamine (PAMAM), for example wherein the amine-based polymer comprises polyethylenimine (PEI) and/or ethoxylated polyethylenimine (PEIE).
4. The wearable sensor according to aspect claim 3 wherein the adhesive composition comprises the amine-based polymer in a range from 1 wt. % to 2 wt. %, preferably in a range from 1.2 wt. % to 1.5 wt. %, by weight of the adhesive polymer composition.
5. The wearable sensor composition according to aspect 4 wherein the amine-based polymer comprises polyethylenimine (PEI), including polyethylenimine PEI-derivatives such as ethoxylated polyethylenimine (PEIE).
6. A wearable sensor according to any preceding aspect wherein the adhesive polymer composition is derived from an adhesive polymer mixture comprising: a silicone polymer; a curing agent; and an amine-based polymer, wherein the adhesive polymer mixture comprises from 1 to 10 wt. % amine-based polymer by weight of the adhesive polymer mixture.
7. The wearable sensor according to aspect 6 wherein the silicone polymer is uncured polydimethylsiloxane and/or the curing agent is a curing agent for polydimethylsiloxane and/or the amine-based polymer is polyethylenimine (PEI), such as ethoxylated polyethylenimine (PEIE).
8. The wearable sensor according to aspect 7 wherein the adhesive polymer mixture comprises less than 10 wt. % of the curing agent by weight of the adhesive composition.
9. The wearable sensor according to any preceding aspect, wherein the adhesive composition forms a film layer on the skin-contacting surface.
10. The wearable sensor according to any preceding aspect, further comprising: at least one electrode pad for monitoring physiological and/or brain signals; and optionally, circuitry electrically coupled to the at least one electrode pad.
11. The wearable sensor according to aspect 10, wherein the at least one electrode pad is at least partially coated in a conductive polymer composition.
12. The wearable sensor according to aspect 11, wherein the conductive polymer composition comprises: an electrically-conductive material; at least one additive; and a silicone polymer.
13. The wearable sensor according to aspect 12, wherein the silicone polymer is polydimethylsiloxane.
14. The wearable sensor according to aspect 12 or 13 wherein the electrically conductive material is particulate carbon.
15. The wearable sensor according to aspect 14 wherein the particulate carbon is in a range from 5 wt. % to 20 wt % by weight of the conductive polymer composition.
16. The wearable sensor according to aspects 12 to 15 wherein the at least one additive comprises a surfactant, for example Triton X-100 and/or Tween 80.
17. The wearable sensor according to aspect 16 wherein the surfactant is Triton X-100, and the Triton X-100 is present in a range from 1 wt. % to 30 wt. %, preferably in a range from 1 wt. % to 20 wt. %, more preferably in a range from 5 wt. % to 15 wt. %, for example about 10 wt. % such as 10 wt. %, by weight of the conductive polymer composition.
18. The wearable sensor according to aspect 16 wherein the surfactant is Tween 80, and the Tween 80 is present in a range from 1 wt. % to 20 wt. %, preferably in a range from 1 wt. % to 10 wt. %, more preferably in a range from 1 wt. % to 5 wt. %, for example about 2.5 wt. % such as 2.51 wt. %, by weight of the conductive polymer composition.
19. The wearable sensor according to aspects 12 to 17 wherein the at least one additive comprises a first additive and a second additive.
20. The wearable sensor according to aspect 19 wherein the second additive is ethylene glycol.
21. The wearable sensor according to aspect 19 wherein the ethylene glycol is in a range from 1 wt. % to 30 wt. %, preferably in a range from 1 wt. % to 20 wt. %, more preferably in a range from 3 wt. % to 15 wt. %, most preferably in a range from 6 wt. % to 8 wt. %, for example about 7 wt. % such as 7 wt. %, by weight of the conductive polymer composition.
22. The wearable sensor according to any preceding aspect wherein at 30 Hz, an electrical skin impedance of the conductive polymer is in a range from 100 kΩ to 2000 kΩ, preferably in a range from 100 kΩ to 1000 kΩ, more preferably in a range from 50 kΩ to 500 kΩ, most preferably in a range from 10 kΩ to 200 kΩ.
23. The wearable sensor according to aspects 11 to 22 wherein a skin-contacting surface of the conductive polymer composition comprises patterning for increasing a surface area of the coating.
24. A method for manufacturing a wearable sensor for monitoring physiological and/or brain signals, the wearable sensor having a flexible substrate and at least one electrode pad, the method comprising: coating at least part of the flexible substrate in an adhesive polymer mixture, the mixture comprising a silicone polymer, an amine-based polymer, and optionally a curing agent, wherein the adhesive polymer mixture comprises from 1 to 10 wt. % amine-based polymer by weight of the adhesive polymer mixture; curing the adhesive polymer mixture to obtain a cured adhesive polymer coating; coating at least part of the at least one electrode pad in a conductive polymer mixture, the mixture comprising an electrically-conductive material, at least one additive, a silicone polymer, and optionally a curing agent; and curing the electrically-conductive polymer mixture to obtain a cured electrically-conductive polymer on the at least one electrode pad.
25. The method according to aspect 24 further comprising: patterning a surface of the electrically-conductive polymer, prior to curing, to increase a surface area of the coating.

While at least one exemplary embodiment of the present invention(s) is disclosed herein, it should be understood that modifications, substitutions and alternatives may be apparent to one of ordinary skill in the art and can be made without departing from the scope of this disclosure. This disclosure is intended to cover any adaptations or variations of the exemplary embodiment(s). In addition, in this disclosure, the terms “comprise” or “comprising” do not exclude other elements or steps, the terms “a” or “one” do not exclude a plural number, and the term “or” means either or both. Furthermore, characteristics or steps which have been described may also be used in combination with other characteristics or steps and in any order unless the disclosure or context suggests otherwise. This disclosure hereby incorporates by reference the complete disclosure of any patent or application from which it claims benefit or priority.

1. A wearable sensor for monitoring physiological, or brain signals, or both, the sensor comprising:
a thin flexible substrate comprising a skin-contacting surface, wherein the skin-contacting surface is at least partially coated in an adhesive polymer composition, the adhesive polymer composition comprising:
a silicone polymer; and
an amine-based polymer,
wherein the adhesive polymer composition comprises from 1 to 10 wt. % amine-based polymer by weight of the adhesive polymer composition.

2. The wearable sensor as claimed in claim 1 wherein the silicone polymer is polydimethylsiloxane.

3. The wearable sensor as claimed in claim 1 wherein the amine-based polymer is selected from one or more of a group consisting of: polyethylenimine, polyethylenimine PEI-derivatives, ethoxylated polyethylenimine (PEIE), aminoethylaminopropyltrimethoxysilane (AEPS) and polyamidoamine (PAMAM).

4. The wearable sensor as claimed in claim 3 wherein the adhesive composition comprises the amine-based polymer in a range from 1 wt. % to 2 wt by weight of the adhesive polymer composition.

5. The wearable sensor as claimed in claim 1 wherein the adhesive polymer composition is derived from an adhesive polymer mixture comprising:
a silicone polymer;
a curing agent; and
an amine-based polymer,
wherein the adhesive polymer mixture comprises from 1 to 10 wt. % amine-based polymer by weight of the adhesive polymer mixture.

6. The wearable sensor as claimed in claim 5 wherein
the silicone polymer is uncured polydimethylsiloxane, or
the curing agent is a curing agent for polydimethylsiloxane, or
the amine-based polymer is polyethylenimine (PEI), or
any combination thereof.

7. The wearable sensor as claimed in claim 6 wherein the adhesive polymer mixture comprises less than 10 wt. % of the curing agent by weight of the adhesive composition.

8. The wearable sensor as claimed in claim 1, wherein the adhesive polymer composition forms a film layer on the skin-contacting surface.

9. The wearable sensor as claimed in claim 1, further comprising:
at least one electrode pad for monitoring physiological and/or brain signals; and
optionally, circuitry electrically coupled to the at least one electrode pad.

10. The wearable sensor as claimed in claim 9, wherein the at least one electrode pad is at least partially coated in a conductive polymer composition.

11. The wearable sensor as claimed in claim 10, wherein the conductive polymer composition comprises:
an electrically-conductive material;
at least one additive; and
a silicone polymer.

12. The wearable sensor as claimed in claim 11, wherein the silicone polymer is polydimethylsiloxane, or
wherein the electrically conductive material is particulate carbon, or both.

13. The wearable sensor as claimed in claim 12 wherein the particulate carbon is in a range from 5 wt. % to 20 wt % by weight of the conductive polymer composition.

14. The wearable sensor as claimed in claim 11 wherein the at least one additive comprises a surfactant.

15. The wearable sensor as claimed in claim 11 wherein the at least one additive comprises a first additive and a second additive.

16. The wearable sensor as claimed in claim 15 wherein the second additive is ethylene glycol.

17. The wearable sensor as claimed in claim 16 wherein the ethylene glycol is in a range from 1 wt. % to 30 wt. % by weight of the conductive polymer composition.

18. The wearable sensor as claimed in claim 1 wherein at 30 Hz, an electrical skin impedance of the adhesive polymer composition is in a range from 100 kΩ to 2000 kΩ.

19. The wearable sensor as claimed in claim 1 wherein a skin-contacting surface of the adhesive polymer composition comprises patterning for increasing a surface area of the coating.

20. A method for manufacturing a wearable sensor for monitoring physiological, or brain signals, or both, the wearable sensor having a flexible substrate and at least one electrode pad, the method comprising:
coating at least part of the flexible substrate in an adhesive polymer mixture, the adhesive polymer mixture comprising a silicone polymer, an amine-based polymer, and optionally a curing agent, wherein the adhesive polymer mixture comprises from 1 to 10 wt. % amine-based polymer by weight of the adhesive polymer mixture;
curing the adhesive polymer mixture to obtain a cured adhesive polymer coating;
coating at least part of the at least one electrode pad in a conductive polymer mixture, the mixture comprising an electrically-conductive material, at least one additive, a silicone polymer, and optionally a curing agent;
curing the electrically-conductive polymer mixture to obtain a cured electrically-conductive polymer on the at least one electrode pad; and
optionally, patterning a surface of the electrically-conductive polymer, prior to curing, to increase a surface area of the coating.
